(19) United States
(12) Patent Application Publication
(10) Pub. No.: US 2018/0007277 A1
(43) Pub. Date: Jan. 4, 2018

(54) EXERCISE EVALUATING APPARATUS

(71) Applicant: CASIO COMPUTER CO., LTD., Tokyo (JP)

(72) Inventor: Takehiro Aibara, Tokyo (JP)

(21) Appl. No.: 15/499,329

(22) Filed: Apr. 27, 2017

(30) Foreign Application Priority Data
Jun. 29, 2016 (JP) 2016-128342
Feb. 3, 2017 (JP) 2017-018198

(51) Int. Cl. H04N 5/232; A61B 5/00; A61B 5/11; H04N 5/265

(52) U.S. Cl. CPC H04N 5/23293; H04N 5/265; A61B 5/7425; A61B 5/1121; A61B 5/0077; A61B 5/1128; A61B 2562/0219; A61B 2576/00

(57) ABSTRACT

An exercise evaluating apparatus including: a processor; and a recording medium which records a program executed by the processor to perform processing, wherein, the processor is configured to perform: generating a combined image from a plurality of images successively imaging a moving subject, the combined image being an image in which a change in motion of the subject according to a time series can be understood; obtaining motion data corresponding to motion of the subject output from a motion sensor attached to the subject; and controlling the display to display the combined image and the motion data in a state corresponding a time series of the plurality of images used in generating the combined image with a time series of the obtained motion data.

### CROSS-REFERENCE TO RELATED APPLICATIONS

This application is based upon and claims the benefit of priority from the prior Japanese Patent Application No. 2016-128342, filed on Jun. 29, 2016, and the prior Japanese Patent Application No. 2017-018198, filed on Feb. 3, 2017 the entire contents of which are incorporated herein by reference.

### BACKGROUND OF THE INVENTION

### 1. Field of the Invention

The present invention relates to an exercise evaluating apparatus, an exercise evaluating method, and a recording medium.

### 2. Description of the Related Art

Conventionally, there is a measuring apparatus which is able to measure data regarding foot-strike timing, toe-off timing, contact time, etc. during walking from the output result of an acceleration sensor in the advancing direction and the perpendicular direction when a human walks with the acceleration sensor attached (Japanese Patent Application Laid-Open Publication No. 2012-179114).

### SUMMARY OF THE INVENTION

According to an aspect of the present invention, there is provided an exercise evaluating apparatus comprising: a processor; and a recording medium which records a program executed by the processor to perform processing, wherein, the processor is configured to perform: generating a combined image from a plurality of images successively imaging a moving subject, the combined image being an image in which a change in motion of the subject according to a time series can be understood; obtaining motion data corresponding to motion of the subject output from a motion sensor attached to the subject; and controlling a display to display the combined image and the motion data in a state corresponding a time series of the plurality of images used in generating the combined image with a time series of the obtained motion data.

### BRIEF DESCRIPTION OF THE DRAWINGS

FIG. 1 is a block diagram showing an exercise evaluating system according to an embodiment of the present invention.

FIG. 2A is a diagram showing a user wearing a measuring apparatus according to the present embodiment.

FIG. 2B is a diagram showing a user wearing a measuring apparatus according to the present embodiment.

FIG. 3A is a block diagram showing a functional configuration of the exercise evaluating apparatus.

FIG. 3B is a block diagram showing a functional configuration of a measuring apparatus.

FIG. 3C is a block diagram showing a functional configuration of an imaging apparatus.

FIG. 4 is a flowchart showing an exercise evaluating process according to embodiment 1.

FIG. 5 is a flowchart showing a display control process.

FIG. 6A is a diagram showing a world coordinate axis.

FIG. 6B is a diagram showing a change in a posture of the user wearing the measuring apparatus.

FIG. 6C is a diagram showing a change in a posture of the user wearing the measuring apparatus.

FIG. 6D is a diagram showing a waveform of acceleration data in a Z-axis, and a waveform of acceleration data in the Z-axis after a smoothing process.

FIG. 6E is a diagram showing an estimating process of a foot-strike position.

FIG. 7 is a diagram showing a waveform of angular speed data with each axis as the center while running.

FIG. 8 is a diagram showing an estimating process of a toe-off position.

FIG. 9 is a diagram showing each phase of a running cycle.

FIG. 10A is a diagram showing an example of exercise analysis data.

FIG. 10B is a diagram showing an example of exercise analysis data.

FIG. 10C is a diagram showing an example of exercise analysis data.

FIG. 11 is a diagram showing an example of a display screen of an exercise evaluating apparatus when an exercise evaluating process is performed.

FIG. 12 is a diagram showing an example of a display screen of an exercise evaluating apparatus when an exercise evaluating process is performed.

FIG. 13 is a diagram showing an example of a display screen of an exercise evaluating apparatus when an exercise evaluating process is performed.

FIG. 14 is a diagram showing an example of a display screen of an exercise evaluating apparatus when an exercise evaluating process is performed.

FIG. 15 is a diagram showing an example of exercise analysis data.

FIG. 16 is a diagram showing an example of a display screen of an exercise evaluating apparatus when an exercise evaluating process is performed.

FIG. 17 is a diagram showing an example of exercise analysis data.

FIG. 18 is a flowchart showing an exercise evaluating process according to embodiment 2.

FIG. 19A is a diagram showing an example of a display screen of an exercise evaluating apparatus when an exercise evaluating process is performed.

FIG. 19B is a diagram showing an example of a display screen of an exercise evaluating apparatus when an exercise evaluating process is performed.

FIG. 20A is a diagram showing an example of a display screen of an exercise evaluating apparatus when an exercise evaluating process is performed.

FIG. 20B is a diagram showing an example of a display screen of an exercise evaluating apparatus when an exercise evaluating process is performed.

### DETAILED DESCRIPTION OF THE PREFERRED EMBODIMENTS

An embodiment of the present invention is described in detail with reference to the drawings. The scope of the present invention is not limited to the illustrated examples.

### Embodiment 1

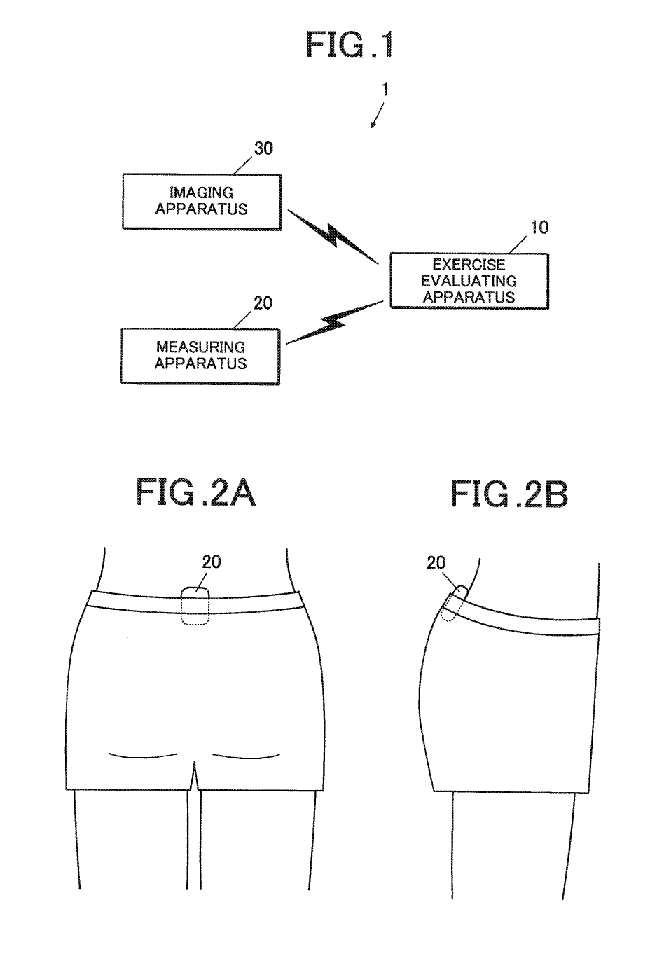

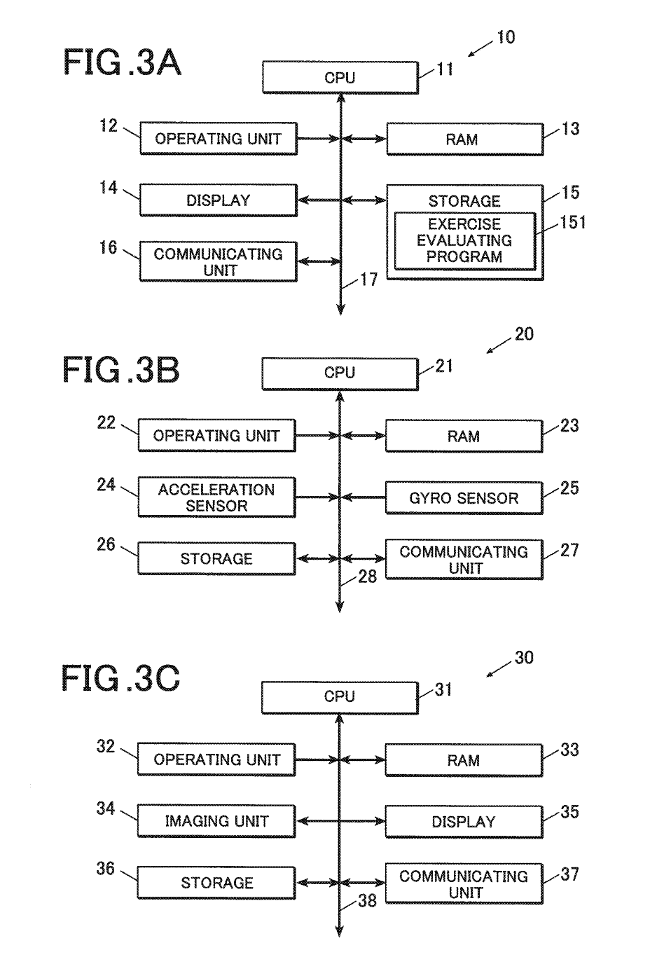

A configuration of an apparatus according to the present embodiment is described with reference to FIG. 1, FIG. 2A to FIG. 2B, and FIG. 3A to FIG. 3C. First, with reference to FIG. 1, an exercise evaluating system 1 of the present embodiment is described. FIG. 1 is a block diagram showing the exercise evaluating system 1 of the present embodiment.

The exercise evaluating system 1 includes an exercise evaluating apparatus 10, a measuring apparatus 20, and an imaging apparatus 30.

The exercise evaluating apparatus 10 obtains motion data from a measuring apparatus 20 worn by a subject and performs exercise analysis. Moreover, the exercise evaluating apparatus 10 obtains an image data regarding running operation of the subject from the imaging apparatus 30. Using such data, the exercise evaluating apparatus 10 generates a multiple exposure combined image in which a plurality of images of the subject in each time span are aligned in order according to the elapsed time. According to an instruction from the user, the exercise evaluating apparatus 10 displays the above analysis result corresponded with the combined image.

As shown in FIG. 2A and FIG. 2B, the measuring apparatus 20 is attached to a position such as the lower back of the subject. The measuring apparatus 20 measures the acceleration and the angular speed in triaxial directions while the subject is running. Here, the left and right direction is to be an X-axis, a front and back direction is to be a Y-axis, and an up and down direction is to be a Z-axis. In the X-axis, the left hand direction is to be positive and the right hand direction is to be negative. In the Y-axis, the direction opposite to the advancing direction is to be positive and the advancing direction is to be negative. In the Z-axis, the upper direction is to be positive and the lower direction is to be negative.

The imaging apparatus 30 is a digital camera to capture the running movement of the subject wearing the measuring apparatus 20.

Next, with reference to FIG. 3A, the internal functional configuration of the exercise evaluating apparatus 10 is described with reference to FIG. 3A. FIG. 3A is a block diagram showing a functional configuration of the exercise evaluating apparatus 10.

As shown in FIG. 3A, the exercise evaluating apparatus 10 includes a CPU (Central Processing Unit) 11 as a generating unit, an exercise analysis unit, a display controller, a first specifying unit, and a second specifying unit, an operating unit 12, a RAM (Random Access Memory) 13, a display 14, a storage 15, and a communicating unit 16. Each unit of the exercise evaluating apparatus 10 is connected to each other through a bus 17.

The CPU 11 controls each unit of the exercise evaluating apparatus 10. The CPU 11 reads a program specified from a system program and application programs stored in the storage 15 and deploys the program in the RAM 13. The CPU 11 performs various processes in coordination with the program.

The operating unit 12 includes a key input unit such as a keyboard and a pointing device such as a mouse. The operating unit 12 receives input of the keys and the positions, and such operation information is output to the CPU 11.

The RAM 13 is a volatile memory and forms a work area to temporarily store various types of data and programs. The display 14 includes a LCD (Liquid Crystal Display), an EL (Electro Luminescence) display, etc., and performs various display according to display information instructed from the CPU 11.

The storage 15 includes a HDD (Hard Disk Drive), etc. and is a storage which can read and write data and programs. Specifically, the storage 15 stores an exercise evaluating program 151.

The communicating unit 16 receives motion data from the measuring apparatus 20 attached to the subject and receives image data regarding the running movement of the subject from the imaging apparatus 30. The communicating unit 16 is a communicating unit which employs a wired method such as a USB terminal or a wireless LAN standard such as Wi-Fi.

When the exercise evaluating apparatus 10 is configured as, for example, a PC (Personal Computer), the operating unit 12 and the display 14 can use an external keyboard and an external monitor, and are not indispensable.

Next, the functional configuration of the measuring apparatus 20 is described with reference to FIG. 3B. FIG. 3B is a block diagram showing a functional configuration of the measuring apparatus 20.

The measuring apparatus 20 includes a CPU 21, an operating unit 22, a RAM 23, an acceleration sensor 24, a gyro sensor 25, a storage 26, and a communicating unit 27. Each unit of the measuring apparatus 20 is connected to each other through a bus 28.

The CPU 21 and the RAM 23 are similar to the CPU 11 and the RAM 13 of the exercise evaluating apparatus 10. The redundant description is omitted, and the different portions are mainly described.

The CPU 21 controls each unit of the measuring apparatus 20. The CPU 21 stores the detecting data (motion data) of the acceleration in each axis output from the acceleration sensor 24 in the storage 26. The CPU 21 stores the detecting data (motion data) of the angular speed with each axis as the center output from the gyro sensor 25 in the storage 26.

The operating unit 22 includes a power button which switches the power ON and OFF, and a start and stop button which instructs start and stop of obtaining data. The buttons are not shown. The CPU 21 controls each unit based on the instruction from the operating unit 22.

The acceleration sensor 24 detects acceleration data in triaxial directions orthogonal to each other. Then, the acceleration sensor 24 outputs the detected acceleration data of each axis to the CPU 21.

The gyro sensor 25 detects the angular speed data with triaxial directions orthogonal to each other as the center. The gyro sensor 25 outputs the detected angular speed data with each axis as the center to the CPU 21. The acceleration data of each axis and the angular speed data with each axis as the center are sampled with a predetermined sampling cycle (for example, 200 Hz).

The storage 26 includes a flash memory, an EEPROM (Electrically Erasable Programmable ROM), etc., and is a storage which is able to read and write data and programs.

The communicating unit 27 outputs acceleration data of each axis stored in the storage 26 and the angular speed data with each axis as the center to the exercise evaluating apparatus 10 according to control by the CPU 21. For example, the communicating unit 27 is a communicating unit which employs a wired method such as a USB terminal or a wireless LAN standard such as Wi-Fi.

Next, the functional configuration of the imaging apparatus 30 is described with reference to FIG. 3C. FIG. 3C is a block diagram showing the functional configuration of the imaging apparatus 30.

The imaging apparatus 30 includes the CPU 31, the operating unit 32, the RAM 33, the imaging unit 34, the display 35, the storage 36, and the communicating unit 37. Each unit of the imaging apparatus 30 is connected to each other through the bus 38.

The CPU 31 and the RAM 33 are similar to the CPU 11 and the RAM 13 of the exercise evaluating apparatus 10. The redundant description is omitted, and the different portions are mainly described.

The CPU 31 controls each unit of the imaging apparatus 30. The CPU 31 stores the image data of the subject (subject wearing the measuring apparatus 20) captured by the imaging unit 34 in the storage 36.

The operating unit 32 includes, for example, a shutter key, a cursor button for moving the cursor up, down, left and right when the operation mode, the function, etc. is selected and instructed, a determination button, and the like. The CPU 31 controls each unit of the imaging apparatus 30 based on the instruction from the operating unit 32. The operating unit 32 can include a touch panel (not shown) provided as one with the display 35.

The imaging unit 34 includes a function to capture the moving image at a predetermined frame rate (for example, 200 fps). For example, the imaging unit 34 captures a series of running movement when the subject runs while wearing the measuring apparatus 20 (at least, the running movement in the term from when one foot touches the ground to when this foot touches the ground again).

The display 35 displays in the display region the image with the predetermined size which is read from the storage 36 and decoded. For example, the display 35 may be a liquid crystal display panel or an organic EL display panel, but this is merely one example, and the display is not limited to the above.

The storage 36 includes a flash memory, an EEPROM (Electrically Erasable Programmable ROM), etc., and is a storage which is able to read and write data and programs.

The communicating unit 37 outputs image data stored in the storage 36 to the exercise evaluating apparatus 10 based on control by the CPU 31. For example, the communicating unit 37 is a communicating unit which employs a wired method such as a USB terminal or a wireless LAN standard such as Wi-Fi.

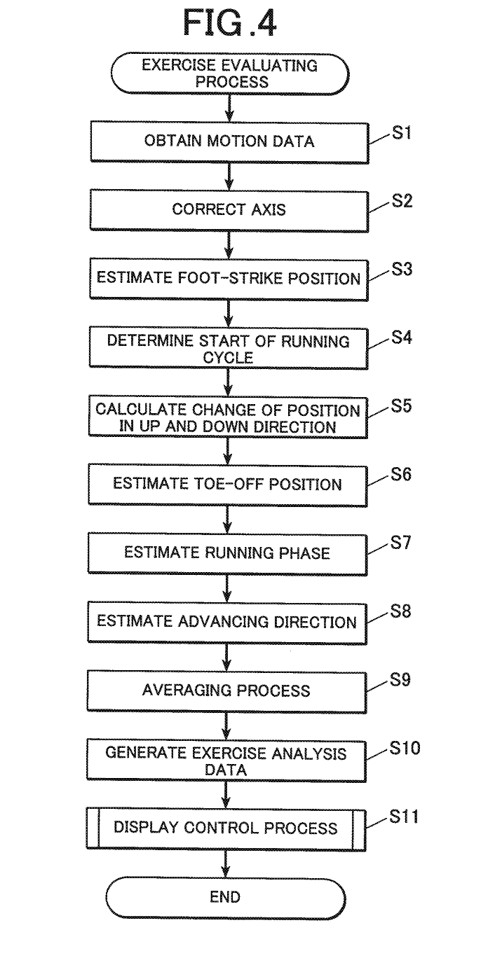

Next, the exercise evaluating process performed by the exercise evaluating apparatus 10 is described with reference to FIG. 4. In the exercise evaluating apparatus 10, when the instruction to perform the exercise evaluating process is input from the user through the operating unit 12, the CPU 11 performs the exercise evaluating process in coordination with the exercise evaluating program 151 read out from the storage 15 and deployed in the RAM 13.

In the exercise evaluating process described below, the motion data and the image data are already stored in the storage 15 before starting the process.

First, the CPU 11 reads out from the storage 15 motion data (acceleration data of each axis and angular speed data with each axis as the center) desired by the user and specified by the user according to operation of the operating unit 12 (step S1).

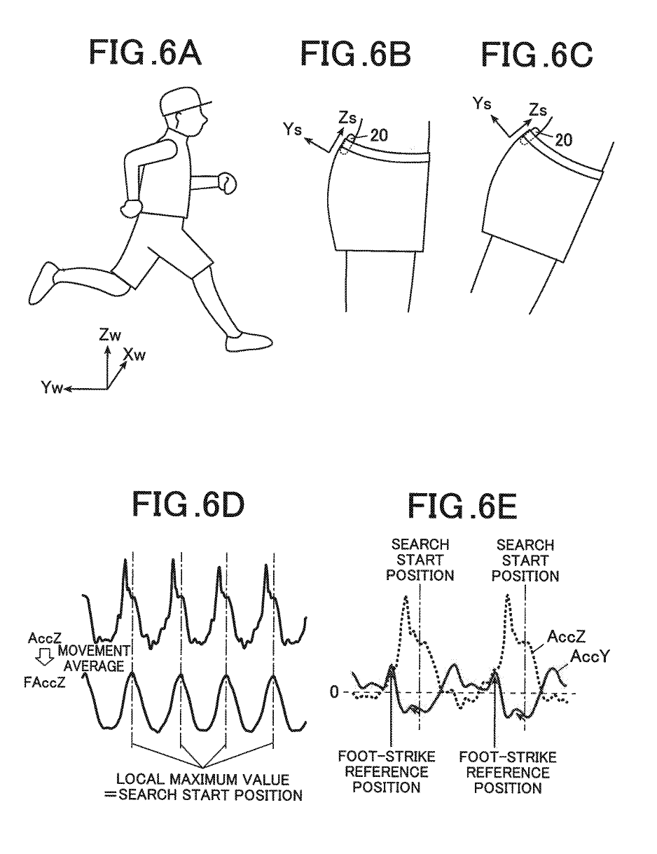

Next, the CPU 11 performs an axis correcting process on the acceleration data of each axis and the angular speed data with each axis as the center (step S2). That is, the CPU 11 converts the above motion data to data using as the reference a world coordinate axis which is constant with respect to the running movement and stores the data (referred to as correction data) in the RAM 13. Here, as shown in FIG. 6A, in the world coordinate axis, the Z-axis (Zw) matches with a gravity direction, the Y-axis (Yw) matches with the direction opposite to the advancing direction of the running, and the X-axis (Xw) matches with the direction perpendicular to the Z-axis and the Y-axis. The reason why the motion data is converted to the data with the world coordinate axis as the reference is because when the subject is wearing the measuring apparatus 20 at the lower back, as shown in FIG. 6B and FIG. 6C, the direction of the Y-axis (Ys) and the Z-axis (Zs) may change depending on the change of posture during running. Specific methods of conversion include, for example, converting the motion data to the data with the world coordinate axis as the reference by estimating the earth axis direction using a Kalman filter and rotating each axis so that the estimated earth axis direction and the Z-axis direction match, or converting the motion data to the data with the world coordinate axis as the reference by estimating the direction of the average acceleration based on the acceleration data in the Z-axis as the earth axis direction and rotating each axis so that the estimated earth axis direction and the Z-axis direction match.

Next, the CPU 11 estimates a foot-strike position where the foot touches the ground based on correction data stored in the RAM 13 (step S3). Specifically, first, the CPU 11 specifies the search start position of the foot-strike position using the Z-axis acceleration data among the correction data. The upper half of FIG. 6D is a graph showing a waveform of the Z-axis acceleration data (AccZ). The waveform of AccZ shows various shapes due to individual difference or speed of running. Since the value in the perpendicular upper direction (plus direction) becomes large due to the shock of striking the ground, the shape becomes a wave drawing a mountain for each step. The CPU 11 uses a smoothing filter such as a moving average, etc. on the Z-axis acceleration data (AccZ) to obtain a waveform of the Z-axis acceleration data (FAccZ) after the smoothing process as shown in the lower half of FIG. 6D. The CPU 11 sets the position showing the local maximum value of the waveform (position from the foot-strike position after a certain amount of time passes) to a search start position to estimate the foot-strike position.

Next, as shown in FIG. 6E, the CPU 11 scans the waveform of the Y-axis acceleration data (AccY) for a certain amount of time on the time axis in the reverse direction from the search start position, and detects the local maximum value. When the foot strikes the ground, a force to stop is applied in a direction rear to the advancing direction (Y-axis plus direction), and the value of AccY largely moves. Therefore, the position of the detected local maximum value is to be a foot-strike reference position. The foot-strike reference position is a peak position of stopping, and occurs a few milliseconds after the actual striking. Therefore, the CPU 11 estimates the position a few milliseconds before the foot-strike reference position as the foot-strike position.

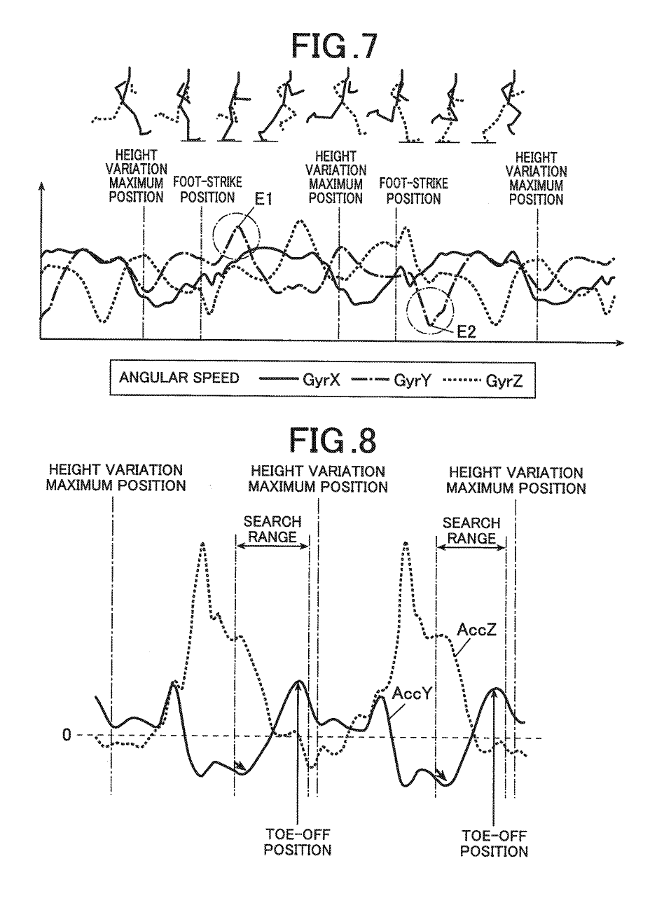

Next, the CPU 11 determines the start of the running cycle (step S4). Specifically, the CPU 11 uses the angular speed data with the Y-axis as the center among the correction data to determine whether the foot striking the ground in the foot-strike position estimated in step S3 is the left foot or the right foot. FIG. 7 is a diagram showing a waveform of the angular speed data with each axis as the center during running, and the foot-strike position estimated in step S3 is shown with a long and short dash line. As shown in FIG. 7, the CPU 11 scans the waveform of the angular speed data with the Y-axis as the center (GyrY) in the range of a certain amount of time (100 milliseconds or less) from the foot-strike position and detects the local value. Then, when the sign of the detected local value E1 is plus, the CPU 11 determines the foot-strike position as the reference of the present detection is a foot-strike by the right foot. On the other hand, when the sign of the detected local value E2 is minus, the CPU 11 determines the foot-strike position as the reference of the present detection is a foot-strike by the left foot. The determining method uses the phenomenon that the pelvis moves downward to the foot opposite of the striking foot and then returns immediately in order to soften the shock of striking the ground. Then, the CPU 11 determines the position determined to be the foot-strike position of the right foot as the start of the running cycle. The CPU 11 may determine the position determined to be the foot-strike position of the left foot as the start of the running cycle.

Next, the CPU 11 calculates the change of the position of the measuring apparatus 20 in the up and down direction, that is, the change in the height of the lower back of the subject (step S5). Specifically, the CPU 11 uses the Z-axis acceleration data among the correction data and performs integration twice on the Z-axis acceleration data to calculate the change in the position of the measuring apparatus 20 in the up and down direction.

Here, the absolute value of the position of the measuring apparatus 20 in the up and down direction is not necessary, and the relative position is enough. Although there is no need to consider the integral constant when performing the integration, error may be accumulated due to the integration. Therefore, it is preferable to calculate using the running cycle attribute. First, regarding the Z-axis acceleration, since the acceleration sensor 24 detects the acceleration in the upper direction while striking, the speed increases in the upper direction by simply integrating. Here, since the running is a cyclic movement, the speed in the up and down direction at the beginning and end of the cycle should be the same. Therefore, by obtaining the average of all data for one running cycle and subtracting the average value from each of the sampled Z-axis acceleration data, gravitational acceleration and noise components can be removed. That is, the integral value of the Z-axis acceleration data for one running cycle is to be 0 in a virtual state without gravitational acceleration. When there is gravitational acceleration and noise, the integral value is considered to show the value of the gravitational acceleration and the value of the noise. Therefore, the process to remove the gravitational acceleration and the noise component is performed. Integration is performed on the Z-axis acceleration data to calculate the Z-axis speed data. Then, integration is performed on the Z-axis speed data to calculate the change in the position of the measuring apparatus 20 in the up and down direction.

Next, the CPU 11 estimates the toe-off position where the foot is raised from the ground based on the correction data stored in the RAM 13 (step S6). When the foot is raised from the ground, the peak value of the Y-axis acceleration data (AccY) occurs because the acceleration element obtained by kicking the ground is lost and the speed decreases. The CPU 11 estimates the position of the peak value as the toe-off position. Specifically, first, the CPU 11 specifies one foot-strike position and obtains the Z-axis displacement data showing the change of the position of the measuring apparatus 20 in the up and down direction from the foot-strike position to one step before the foot-strike position. Then, the CPU 11 detects the maximum value of the Z-axis displacement data and sets the position of the maximum value as the height variation maximum position (see FIG. 8). Next, as shown in FIG. 8, the CPU 11 sets the search range of the toe-off position to be 60% to 95% of the range from the height variation maximum position obtained one step before the foot-strike position to the height variation maximum position obtained with the foot-strike position as the reference. Then, the CPU 11 scans the waveform of the Y-axis acceleration data (AccY) in the search range and estimates the position of the first detected local maximum value as the toe-off position.

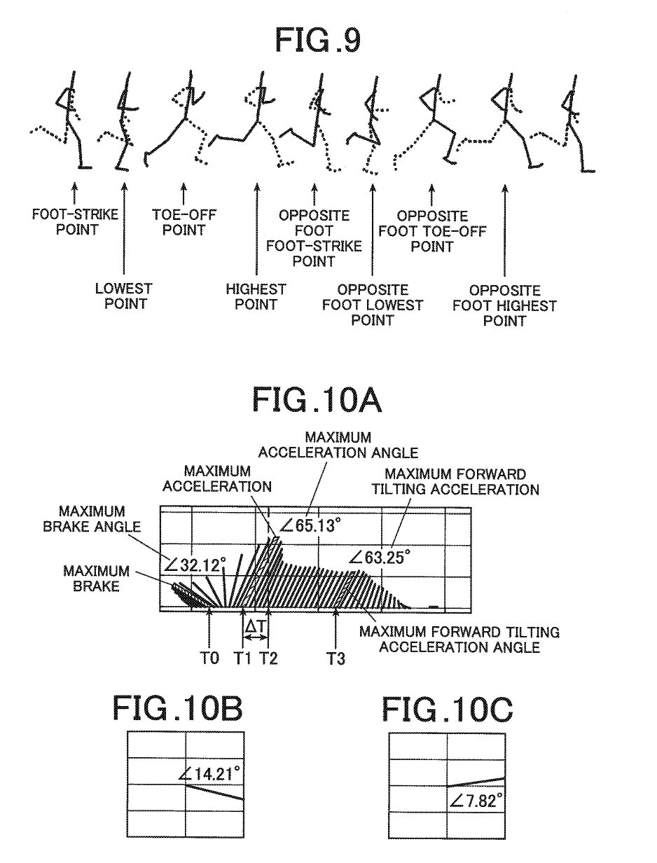

Next, the CPU 11 estimates each phase of the running cycle based on the estimate process of the foot-strike position performed in step S3, the determining process of the start of the running cycle performed in step S4, the calculating process of the position of the positioning apparatus 20 in the up and down direction performed in step S5, and the estimating process of the toe-off position performed in step S6 (step S7). Here, the phases of the running cycle are, as shown in FIG. 9, foot-strike point when the right foot touches the ground, lowest point when the measuring apparatus 20 or the lower back passes the lowest position while the foot touches the ground, toe-off point when the foot is raised from the ground and highest point when the measuring apparatus 20 passes the highest position after the foot is raised from the ground, and opposite foot foot-strike point when the opposite foot (left foot) touches the ground, opposite foot lowest point when the measuring apparatus 20 passes the lowest position while the foot touches the ground, opposite foot toe-off point when the foot is raised from the ground, and opposite foot highest point when the measuring apparatus 20 passes the highest position after the foot is raised from the ground.

Next, the CPU 11 estimates the advancing direction of the running (step S8). Specifically, the CPU 11 performs integration on the angular speed data with the Z-axis as the center, and estimates the average value as the front direction of the body, that is, the advancing direction of running. Here, there is an offset which changes according to time in the angular speed data. Preferably, in order to resolve the offset, before performing the integration on the angular speed data, the average of the sampled angular speed data for one running cycle is obtained, and the integration is performed after subtracting the average value from the original data. The average of the angular speed data is not limited to one running cycle and a few cycles including the cycles before and after can be the target.

Next, the CPU 11 performs an averaging process to reduce the noise of the correction data (triaxial acceleration data) (step S9). Specifically, first, the CPU 11 sets the acceleration data of each axis as the target and converts (normalizes) the time of one running cycle to 400 points. That is, the start of each cycle is 0 point, and the end of each cycle is to be 399 points. Next, the CPU 11 generates the waveform averaging the acceleration data of each cycle (average waveform) and obtains the difference between the waveform of each cycle and the average waveform from 0 to 399 points. The CPU 11 obtains the mean square, and obtains the difference from the average of the cycle by adding the mean square from 0 to 399 points. Then, the CPU 11 sorts the order of each cycle from the difference which is small and selects the wave form of the number used in the averaging process. For example, when the average of 10 waveforms is obtained, 10 waveforms from the top of the sorted waveforms are selected. Then, the CPU 11 obtains the cycle of the selected waveform and calculates the average point number (cyc). The CPU 11 obtains the time from the left foot foot-strike to the right foot foot-strike and calculates the average point number (cyc_L). The CPU 11 also obtains the time from the right foot foot-strike to the left foot foot-strike and calculates the average point number (cyc_R).

Then, the CPU 11 calculates the left foot point number (round_L)=400*cyc_L/cyc and the right foot point number (round_R)=400*cyc_R/cyc. Then, the CPU 11 connects the calculated left point number (round_L) and the calculated right point number (round_R), and obtains the averaged acceleration data for one running cycle.

When the above-described averaging process is performed, the average of the cycle before normalizing (actual cycle time) can be obtained and a cycle which is shifted a certain cycle or more from this average may not be added to this average, or the average of the time from one foot foot-strike to the other foot foot-strike can be obtained, and the cycle which is shifted a certain cycle or more from this average may not be added to this average.

Then, the CPU 11 performs integration on the averaged acceleration data of each axis with the normalized sample interval Δt, and calculates the speed data of each axis. Here, the average of all data for one cycle is obtained, and the average value is subtracted from each sampled acceleration to perform the integration. The CPU 11 performs integration on the speed data of each axis obtained by integration to obtain displacement data of each axis.

In step S9, the CPU 11 converts the time of each phase of running estimated in step S7 to a normalized time of 0 to 399 points.

Next, the CPU 11 generates exercise analysis data as shown in FIG. 10A based on data averaged in step S9 and information of normalized time of each phase in running (step S10).

The exercise analysis data shown in FIG. 10A is data showing time in the horizontal axis, and the time of each sampling point in the origin. The exercise analysis data is data drawing the combined vector combining the Z-axis acceleration vector (vertical direction) and Y-axis acceleration vector (horizontal direction). That is, the change of the combined vector for each elapsed amount of time is shown, and the length of the line showing the combined vector shows the magnitude of the acceleration. The exercise analysis data shown in FIG. 10A shows only data from the right foot foot-strike phase at a certain timing to the right foot toe-off phase.

As shown in FIG. 10A, the exercise analysis data shows the maximum acceleration, the maximum acceleration angle and the maximum acceleration occurring time T1 as the indexes.

The above indexes can be obtained by the following. That is, the sampling data of the period which is from the foot-strike phase to the toe-off phase and in which the value of the Y-axis acceleration data is minus, that is, the period which is accelerating in the advancing direction is extracted, the square of the value of the Y-axis acceleration data and the square of the value of the Z-axis acceleration data are added for each of the sampling data to obtain the square root. With this, the magnitude of the acceleration on the YZ plane is obtained. The time that the magnitude of the acceleration becomes the largest is to be the maximum acceleration occurring time. The magnitude of the acceleration is to be the maximum acceleration. The angle between the combined vector and the Y-axis is to be the maximum acceleration angle. According to the above example, the maximum acceleration on the YZ plane is obtained, but alternatively, the X-axis acceleration data can be considered to obtain the maximum acceleration in three dimensions.

As shown in FIG. 10A, the exercise analysis data shows the time difference ΔT between the maximum acceleration occurring time T1 and the lowest point T2 as the index. The index subtracts the maximum acceleration occurring time from the lowest point time and is an important index for exercise analysis.

As shown in FIG. 10A, the exercise analysis data shows a maximum brake, a maximum brake angle and a maximum brake occurring time T0 as indexes.

Regarding these indexes, the sampling data of the period which is from the foot-strike phase to the toe-off phase and in which the value of the Y-axis acceleration data is plus, that is, the period which is accelerating in the direction opposite to the advancing direction is extracted, the square of the value of the Y-axis acceleration data and the square of the value of the Z-axis acceleration data are added for each of the sampling data to obtain the square root. With this, the magnitude of the acceleration on the YZ plane is obtained. The time that the magnitude of the acceleration becomes the largest is to be the maximum brake occurring time. The magnitude of the acceleration is to be the maximum brake. The angle between the combined vector and the Y-axis is to be the maximum brake angle. According to the above example, the maximum brake on the YZ plane is obtained, but alternatively, the X-axis acceleration data can be considered to obtain the maximum brake in three dimensions. Further, the value of time integration of the Y-axis acceleration of the period accelerating in the direction opposite of the advancing direction between the period from the foot-strike phase and the toe-off phase is obtained as the total brake and this can be added to the index.

As shown in FIG. 10A, the exercise analysis data shows a maximum forward tilting speed, a maximum forward tilting speed angle, and a maximum forward tilting speed occurring time T3 as indexes.

Regarding these indexes, the sampling data of the period which is from the foot-strike phase to the toe-off phase and in which the value of the Y-axis acceleration data is minus, that is, the period which is accelerating in the advancing direction is extracted, and the angle between the combined vector and the Y-axis is obtained for each of the sampling data. Among the above, the time when the angle between combined vector and the Y-axis is smallest (most forward tilt) is to be the maximum forward tilting speed occurring time. The magnitude of the acceleration is to be the maximum forward tilting acceleration. The angle between the combined vector and the Y-axis is to be the maximum forward tilting angle.

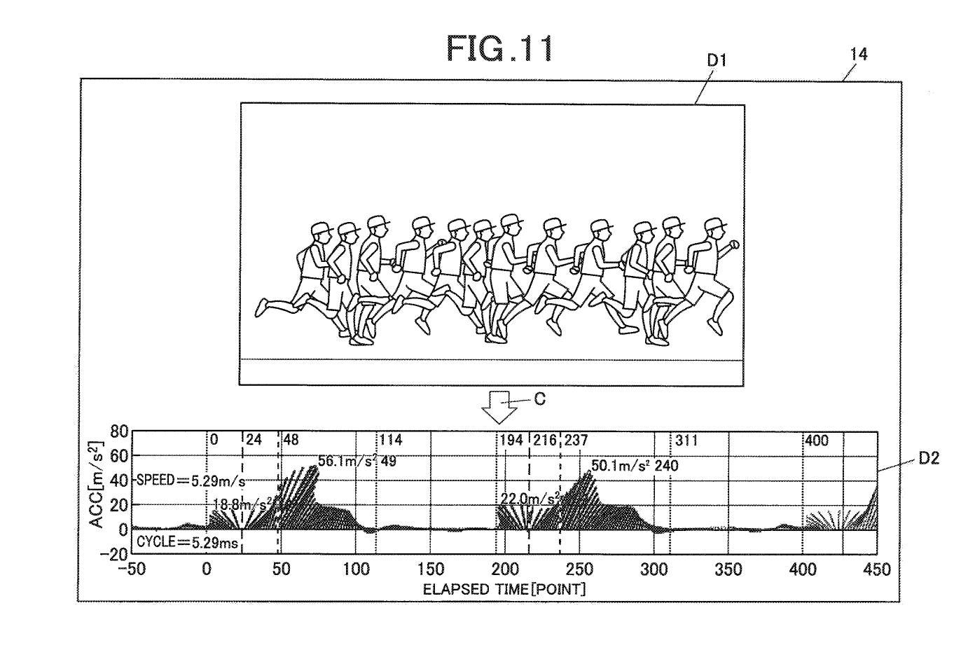

Next, as shown in FIG. 11, the CPU 11 performs display control process to display on the display 14 an exercise analysis result D2 of the time span desired by the user and a combined image D1 corresponding to the time span among the exercise analysis data generated in step S10 (step S11), and ends the exercise evaluating process. The details of the display control process are described in detail later.

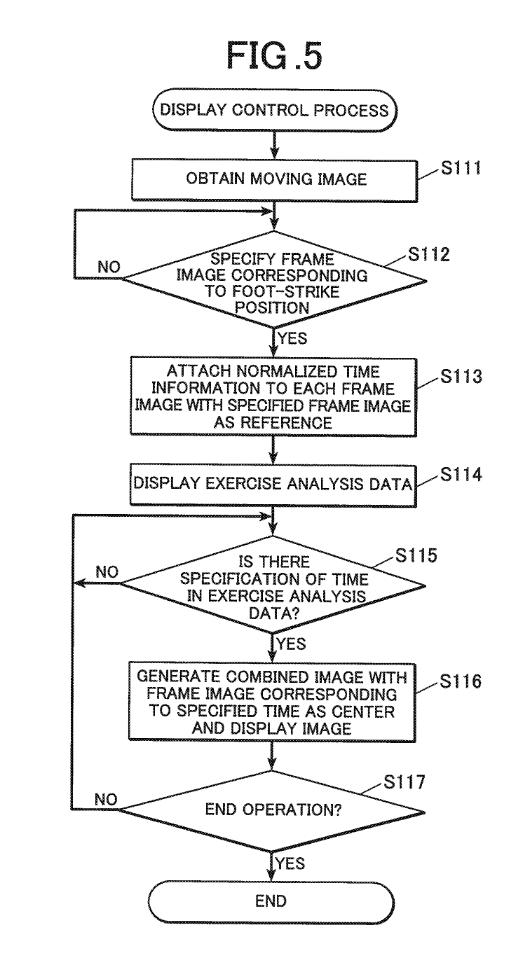

Next, the display control process performed in the exercise evaluating apparatus 10 is described with reference to FIG. 5.

First, the CPU 11 reads the image data specified by the user on the operating unit 12 from the storage 15 (step S111). The image data is image data of the moving image regarding the running captured while obtaining motion data read in step S1.

Next, the CPU 11 determines whether the user operated the operating unit 12 to specify which frame image among the frame images included in the above image data corresponds to the foot-strike position (step S112).

In step S112, when it is determined that the foot-strike position is not specified (step S112; NO), the determining process of step S112 is performed until the specification is made.

Then, when it is determined that the foot-strike position is specified (step S112; YES), the CPU 11 attaches information of the normalized time to each frame image with the specified frame image as the reference (step S113).

Here, the method of attaching the information of the normalized time on each frame image in the term from 0 to 400 points among the information of the normalized time is described. In the averaging process shown in step S9, in the above term, the conversion is made so that the foot-strike position of the left foot is 0 points, the foot-strike position of the right foot is 193 points, and the next foot-strike position of the left foot is 400 points.

For example, when the user specifies that the foot-strike position of the left foot is in the 100-th frame, the foot-strike position of the right foot is in the 145-th frame, and the next foot-strike position of the left foot is in the 200-th frame, the interval between frame images from the 100-th frame to the 145-th frame is a value dividing 193 points by 45 (=145−100). Therefore, for example, the information of the normalized time of the 120-th frame is to be 86 points (≅(120−100)*193/45).

The interval of the frame images from the 145-th frame to the 200-th frame is a value dividing 207 points (=400-193) by 55 (=200-145). Therefore, for example, the information of the normalized time of the 180-th frame is to be 325 points (≅((180-145)*207/55)+193).

Next, as shown in FIG. 11, the CPU 11 displays the exercise analysis result D2 generated in step S10 in the bottom of the display 14 (step S114). Here, the 0 point position of the exercise analysis result D2 shows the left foot foot-strike position. The 27 point position shows the acceleration switching position in which the movement changes from decelerating to accelerating. The 55 point position shows the lowest point position. The 107 point position shows the left foot toe-off position. The 193 point position shows the right foot foot-strike position. The 219 point position shows the acceleration switching position in which the movement changes from decelerating to accelerating. The 249 point position shows the lowest point position. The 304 point position shows the right foot toe-off position. The 400 point position shows the left foot foot-strike position again. That is, the 0 point position to 399 point position shows one running cycle. In the exercise analysis result D2, the speed value shows the average speed of the analyzed period, the cycle value shows the time of one running cycle in the analyzed period, that is, the actual time between 0 point to 399 points, and the values such as 56.1 m/s2, 49, etc. show the magnitude of the maximum acceleration and the timing (point position) that this occurs. As described above, the exercise analysis result D2 shows the direction and the magnitude of the acceleration vector for each elapsed time, and the length of the line shows the magnitude of the acceleration. The value of the vertical axis shown in FIG. 11 shows the magnitude of the acceleration when the direction of the combined vector is the vertical direction.

Then, the CPU 11 determines whether the time in the exercise analysis result D2 (elapsed time) is specified (step S115). Here, the specifying operation of the time in the exercise analysis result D2 (elapsed time) is described. First, the user operates the operating unit 12 and specifies the display range of the exercise analysis result D2. The exercise analysis result D2 can be slid in the left direction or the right direction by specifying. Then, as shown in FIG. 11, a fixed cursor C is displayed in a certain position in the middle of the display 14, and the desired time (elapsed time) is specified by sliding the exercise analysis result D2 so that the position of the desired time (elapsed time) matches the position of the fixed cursor C. The display shown in FIG. 11 shows the right foot foot-strike position (elapsed time; 193 points) matched to the position of the fixed cursor C.

In step S115, when it is determined that the time in the exercise analysis result D2 (elapsed time) is not specified (step S115; NO), the determining process of step S115 is performed until the specification is made.

Then, when it is determined that the time in the exercise analysis result D2 (elapsed time) is specified (step S115; YES), the CPU 11 sets the frame image corresponding to the specified time as a central image, extracts a plurality of frame images (for example, 12 images) at a predetermined interval with the central image as the reference, and generates the combined image D1 combining the above plurality of frame images. As shown in FIG. 11, the CPU 11 displays the generated combined image D1 in the upper side of the display 14 (step S116).

Next, the CPU 11 determines whether the user operated the operating unit 12 to end the display control process (step S117).

In step S117, when it is determined that the operation to end the display control process is not performed (step S117; NO), the process advances to step S115.

In step S117, when it is determined that the operation to end the display control process is performed (step S117; YES), the display control process ends.

The present embodiment is described above, but the present invention is not limited to the embodiments described above, and various modifications are possible without leaving the scope of the present invention.

For example, according to the above-described embodiment, the time information is normalized so that the first foot-strike position (left foot foot-strike position) is to be the 0 point position, and the next left foot foot-strike position is to be the 400 point position. However, the value of the time information in normalizing is merely one example, and the present invention is not limited to the above.

According to the above-described embodiment, the transparency of the main subject in the combined image D1 can be reduced or the transparency of the subject can be made higher as the distance from the center becomes farther.

According to the above-described embodiment, as described in step S112 in the display control process (see FIG. 5), the specifying of the frame image corresponding to the foot-strike position is performed by the user operating the operating unit 12. Alternatively, the frame image corresponding to the foot-strike position can be specified by automatically detecting the foot-strike position by image processing.

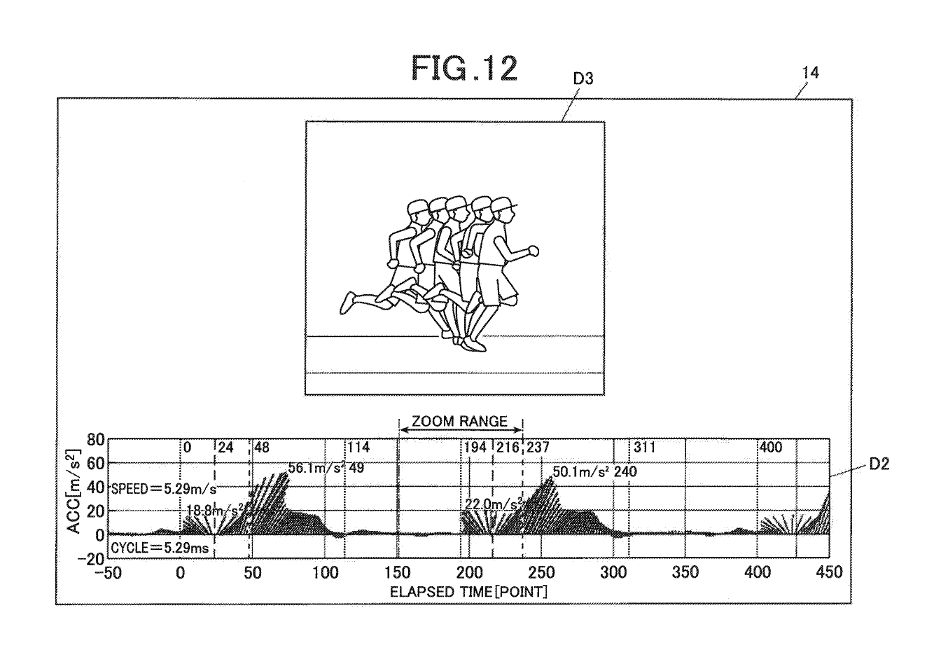

According to the above-described embodiment, the combined image D1 is generated by specifying the desired time (elapsed time) of the exercise analysis result D2, but the present embodiment is not limited to the above. For example, as shown in FIG. 12, a zoom range can be specified with the time (for example, 193 points) specifying the desired time (elapsed time) of the exercise analysis result D2 as the center. Then, the frame image in the zoom range is selected with a certain interval, and the combined image D3 can be generated based on the zoom image zooming on the frame images. The number of frame images selected from the above zoom range can be changed according to the size of the zoom range, or the number of selected frame images can be preset regardless of the zoom range and the frame images can be selected according to the number of frame images set so that the interval becomes even in the zoom range.

According to the above-described embodiment, the exercise analysis data D2 is a diagram showing time in the horizontal axis, the time of each sampling point in the origin, and drawing the combined vector combining the Z-axis acceleration vector (vertical direction) and the Y-axis acceleration vector (horizontal direction). In addition to the above, a foot-strike angle (see FIG. 10B), and a toe-off angle (see FIG. 10C) can be further displayed. The foot-strike angle can be obtained by the Y-axis speed and the Z-axis speed considering the average speed in the foot-strike position. The toe-off angle can be obtained by the Y-axis speed and the Z-axis speed considering the average speed in the toe-off position.

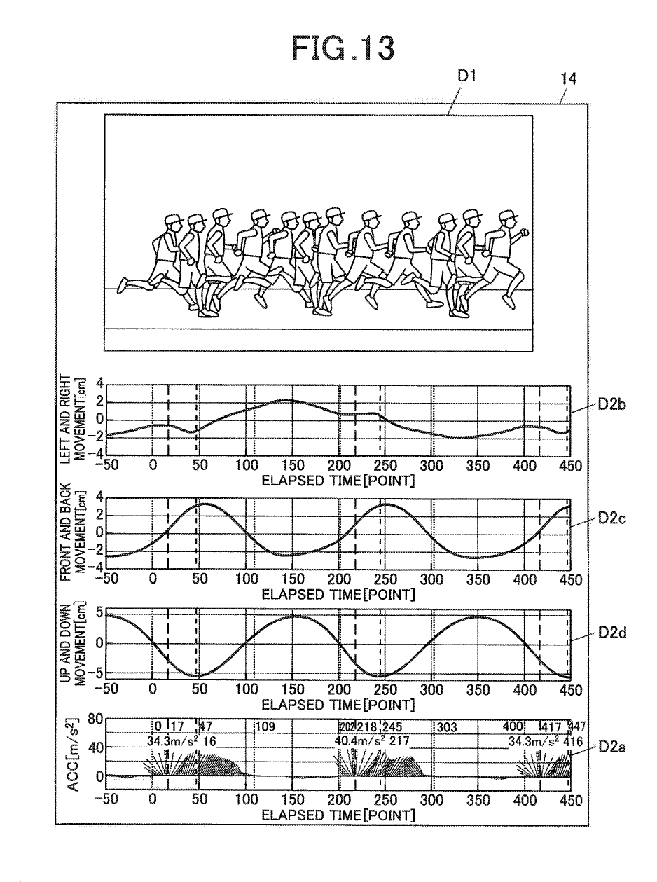

For example, according to the above-described embodiment, as shown in FIG. 13, in addition to the exercise analysis result D2a drawing the above combined vector, an entire or a portion of a X-axis (left and right direction) displacement graph D2b, a Y-axis (front and back direction) displacement graph D2c, a Z-axis (up and down direction) displacement graph D2d can be displayed on the display 14. With this, the displacement of the position of the lower back where the measuring apparatus 20 is attached can be confirmed.

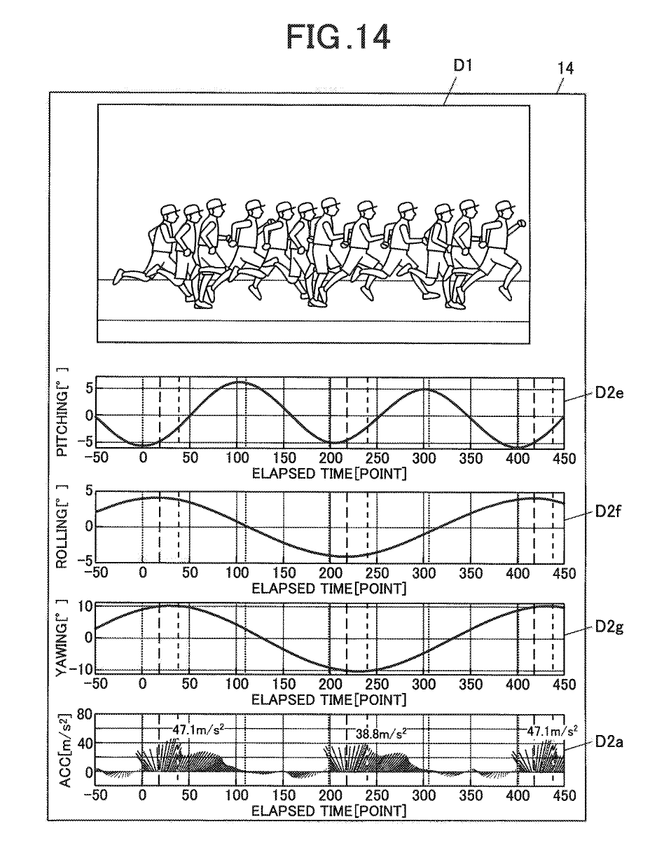

As shown in FIG. 14, in addition to the exercise analysis result D2a drawing the above combined vector, an entire or a portion of a X-axis rotating angle (pitch angle) graph D2e, a Y-axis rotating angle (roll angle) graph D2f, a Z-axis rotating angle (yaw angle) graph D2g can be displayed on the display 14. With this, the rotating amount of the lower back where the measuring apparatus 20 is attached can be confirmed.

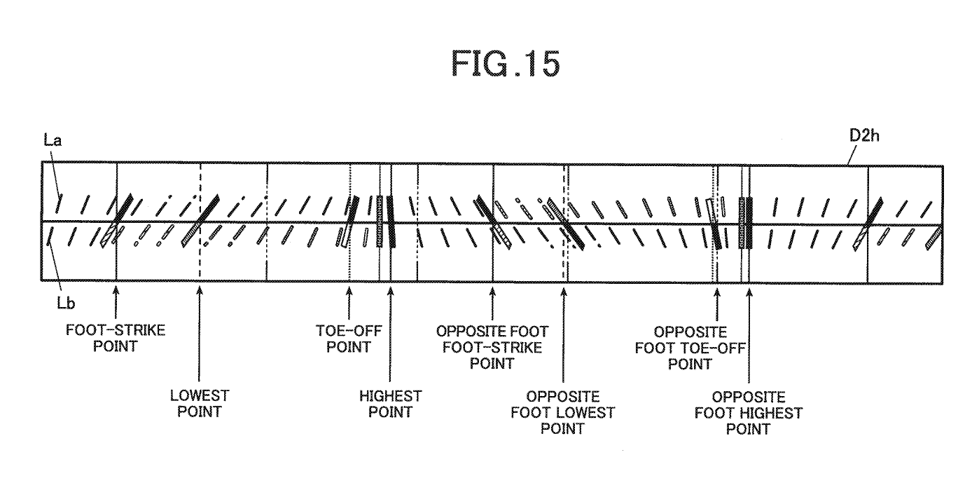

As shown in FIG. 15, the exercise analysis result D2h showing an overlooking view of the position of the lower back during running can be displayed on the display 14 by processing the Z-axis rotating angle (yaw angle) data D2g r. In the exercise analysis result D2h, the lines La shown in the upper side represent the left foot and the lines Lb shown in the lower side represent the right foot. The diagram shows running from the left to the right. Such exercise analysis result D2h and the above-described X-axis (left and right direction) displacement graph D2B can be displayed combined on the display 14. Specifically, the line of the graph shown by the X-axis displacement graph D2b is to be the center of the exercise analysis result D2h (middle point between line La and line Lb) to display both of the above.

According to the above-described embodiment, the combined image D1 and the exercise analysis result D2 are displayed on the display 14 with one subject as the target but the present embodiment is not limited to the above.

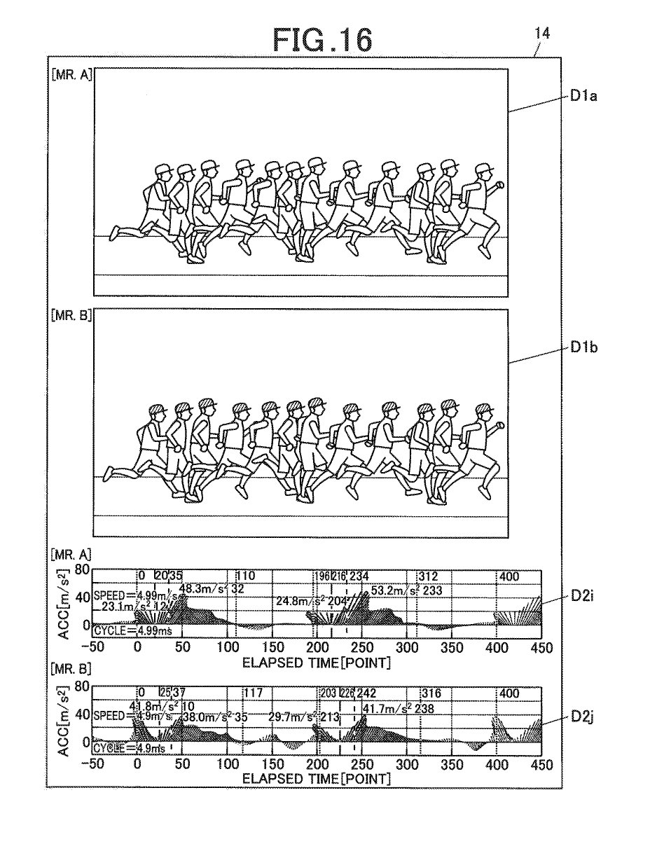

For example, as shown in FIG. 16, two subjects (Mr. A and Mr. B) can be the target, and the combined image D1a for Mr. A and the combined image D1b for Mr. B, and the exercise analysis result D2i for Mr. A and the exercise analysis result D2j for Mr. B can be displayed on the display 14. In this case, the combined image of another person can be compared with the combined image of the user and the exercise analysis result of another person can be compared with the exercise analysis result of the user. With this, the user is able to understand the good points and bad points compared to running movement of other people.

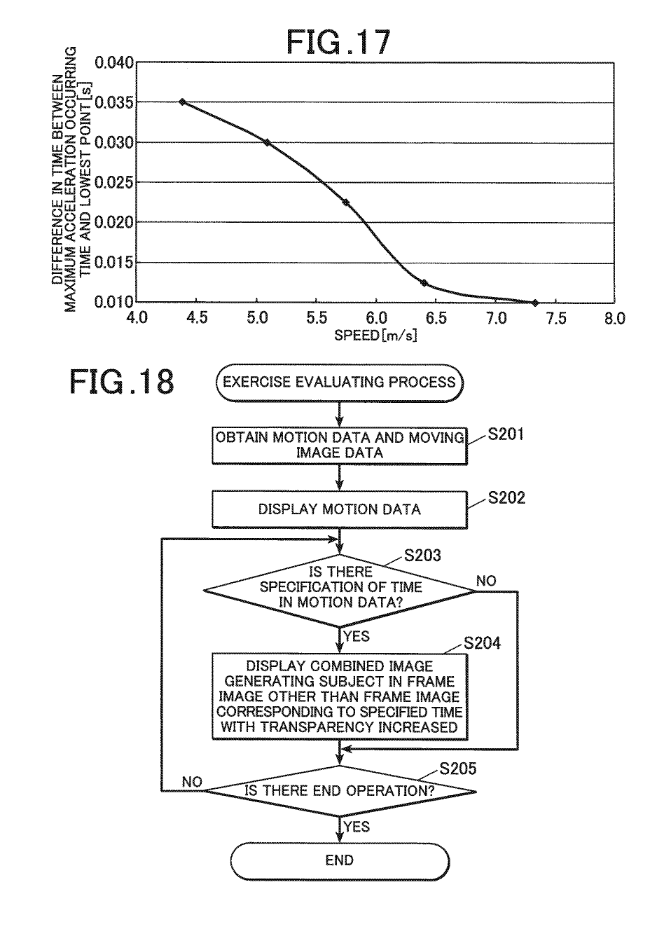

According to the above-described embodiment, in addition to the exercise analysis result D2a drawing the combined vector, as shown in FIG. 17, a graph showing the speed in the horizontal axis and the time difference between the maximum acceleration occurring time and the lowest point in the vertical axis can be displayed on the display 14. The indexes set in the horizontal axis and the vertical axis are not limited to those described above, and indexes regarding the exercise analysis result D2a can be set freely (maximum acceleration, maximum acceleration angle, maximum acceleration occurring time, etc., see FIG. 10A).

According to the above-described embodiment, the combined image is generated based on the plurality of frame images included in the moving image, but the present embodiment is not limited to the above. For example, the combined image can be generated based on the plurality of images captured successively with a high speed at a certain time interval.

According to the above-described embodiment, the combined image D1 displayed in the upper half of the display 14 is displayed in the term cutting out a portion of the exercise analysis result D2 displayed in the lower half of the display 14. That is, the time corresponding to the width of the combined image D1 is the time cutting out a portion of the time corresponding to the width of the exercise analysis result D2. Alternatively, the time corresponding to the width of the combined image D1 and the time corresponding to the width of the exercise analysis result D2 displayed in the lower half can be displayed to match with each other on the display 14. In this case, the position of the combined image D1 corresponding to the position of the fixed cursor C showing the specified time (elapsed time) specified in the exercise analysis result D2 is to be the position of the subject in the specified time (elapsed time). With this, it is possible to omit the process of positioning the subject corresponding to the specified time (elapsed time) in the central position of the horizontal axis of the combined image D1.

### Embodiment 2

Next, the embodiment 2 is described. The same reference numerals are applied to the components similar to the embodiment 1 and the description is omitted.

The exercise evaluating apparatus 10 of embodiment 2 is different from embodiment 1 in the following points, that is, the motion data itself obtained from the measuring apparatus 20 is displayed, and the exercise without movement of the subject is the target and the image data regarding such exercise is combined and displayed.

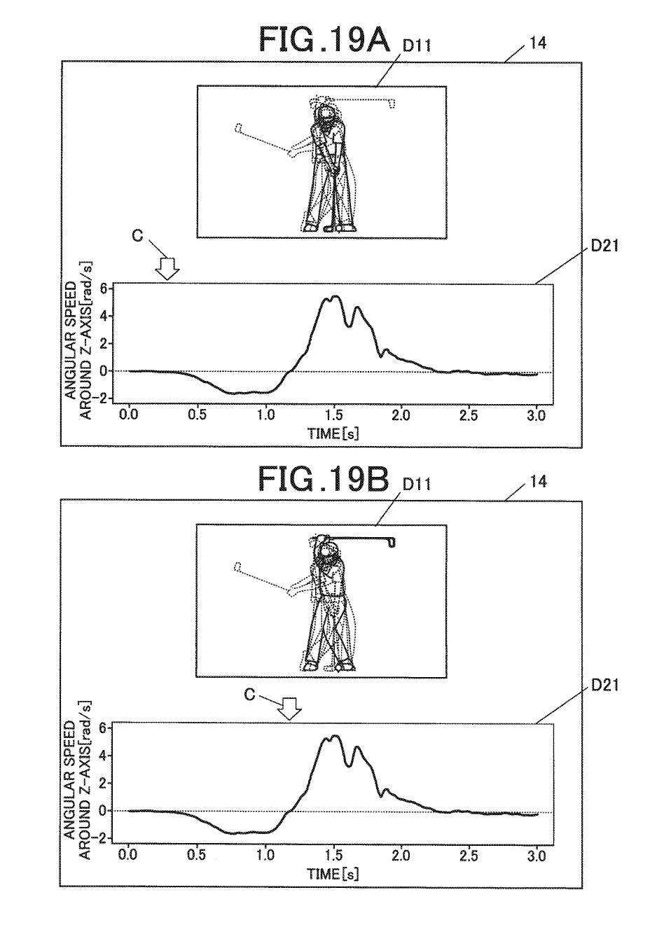

Below, the exercise evaluating apparatus 10 of embodiment 2 is described with reference to FIG. 18 and FIG. 19A to FIG. 19B. FIG. 18 is a flowchart showing the exercise evaluating process performed in the exercise evaluating apparatus 10 of embodiment 2. FIG. 19A and FIG. 19B are diagrams showing an example of the display screen displayed on the display 14 of the exercise evaluating apparatus 10 when the exercise evaluating process is performed with the string of golf swings as the target.

First, the CPU 11 reads from the storage 15 the predetermined motion data (angular speed data with the Z-axis as the center) specified by the user on the operating unit 12 and reads from the storage 15 the image data of the moving image regarding the series of golf swing movements captured while collecting the motion data.

Next, as shown in FIG. 19A and FIG. 19B, the CPU 11 displays the read motion data D21 at the lower side of the display 14 (step S202). Then, the CPU 11 determines whether the time in the motion data D21 (elapsed time) is specified (step S203). Here, as shown in FIG. 19A and FIG. 19B, the desired time is specified in the motion data D21 by the user operating the operating unit 12 to slide the display so that the position of the cursor C displayed in the middle of the display 14 matches the position of the desired time (elapsed time).

In step S203, when it is determined that the time in the motion data D21 (elapsed time) is specified (step S203; YES), the CPU 11 displays in the upper side of the display 14 the combined image D11 (multiple exposure combined image) generated with the transparency of the subject increased in the frame image other than the frame image corresponding to the specified time (step S204). Specifically, for example, when time corresponding to the address of the golf swing is specified, as shown in FIG. 19A, the combined image D11 generated with the transparency of the subject at the point of the address set to “0” and the transparency of the subject at the other points increased is displayed in the upper side of the display 14. In the diagram, the subject at the point of the address is shown with a solid line, and the subject at the other points is shown with a dotted line. Alternatively, for example when time corresponding to the top of the golf swing is specified, as shown in FIG. 19B, the combined image D11 generated with the transparency of the subject at the point of the top set to “0” and the transparency of the subject at the other points increased is displayed in the upper side of the display 14. In the diagram, the subject at the point of the top is shown with a solid line, and the subject at the other points is shown with a dotted line.

In step S203, when it is determined that the time (elapsed time) in the motion data D21 is not specified (step S203; NO), the process of step S204 is skipped, and the process advances to step S205. In this case, the time (elapsed time) is set to a default value, for example 0.0 [s], and the combined image D11 generated with the transparency of the subject of the frame image corresponding to 0.0 [s] as “0”, and with the transparency of the subject in the other points increased is displayed in the upper side of the display 14.

Next, the CPU 11 determines whether the user operated the operating unit 12 to end the exercise evaluating process (step S205).

In step S205, when it is determined that the operation to end the exercise evaluating process is not performed (step S205; NO), the process advances to step S203.

In step S205, when it is determined that the operation to end the exercise evaluating process is performed (step S205; YES), the exercise evaluating process ends.

Embodiment 2 of the present invention is described above, but the present invention is not limited to the above embodiment, and various modifications can be made without leaving the scope of the invention.

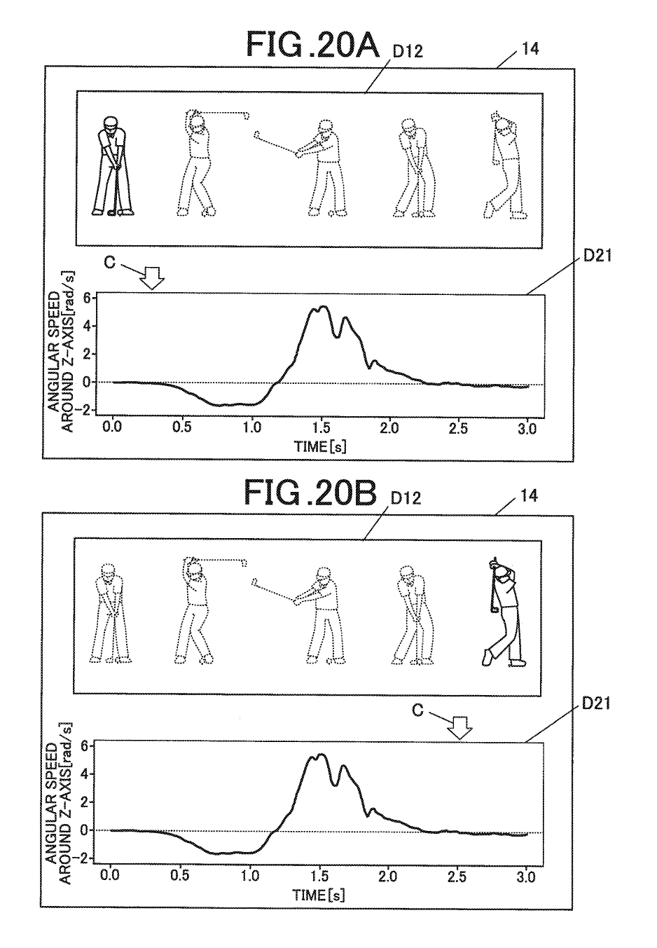

For example, the combined image displayed in the upper side of the display 14 in the exercise evaluating process of the present embodiment can be a combined image 12 aligning the frame images of the subject in a series of a golf swing motion according to the elapsed time as shown in FIG. 20A and FIG. 20B.

A golf swing is provided as an example of the target of the exercise evaluating process in embodiment 2, but other exercise in which the subject does not move can be the target, for example, running using a tread mill or a batting swing in baseball.

According to the exercise evaluating process of the present embodiment, the combined image D11 is displayed in the upper portion of the display 14 and the motion data D21 is displayed in the lower portion of the display 14. Instead of or in addition to the motion data D21, the result of the exercise analysis analyzed base on the motion data D21 can be displayed.

According to the embodiment 1, the combined image D1 is displayed in the upper portion of the display 14 and the exercise analysis result D2 is displayed in the lower portion of the display 14. Instead of or in addition to the exercise analysis result D2, the motion data itself, which is the basis of the exercise analysis, can be displayed.

According to the Embodiments 1 and 2, the user operates the position of the fixed cursor C with the operating unit 2 but the position of the cursor can be automatically moved from the start time to the end time.

The embodiment of the present invention are described above, but the scope of the present invention is not limited to the above-described embodiment. The scope of the present invention is limited to the invention as claimed and its equivalents.

What is claimed is:

1. An exercise evaluating apparatus comprising:
a processor; and
a recording medium which records a program executed by the processor to perform processing,
wherein, the processor is configured to perform:
generating a combined image from a plurality of images successively imaging a moving subject, the combined image being an image in which a change in motion of the subject according to a time series can be understood;
obtaining motion data corresponding to motion of the subject output from a motion sensor attached to the subject; and
controlling a display to display the combined image and the motion data in a state corresponding a time series of the plurality of images used in generating the combined image with a time series of the obtained motion data.

2. The exercise evaluating apparatus according to claim 1, wherein, in the controlling of the display, when the motion data for a predetermined term is displayed on the display, the combined image and the motion data are displayed on the display so that the time series of the plurality of images used in generating the combined image imaged for the predetermined term is corresponded with the time series of the motion data.

3. The exercise evaluating apparatus according to claim 1, wherein,
in the generating, the combined image is generated aligning according to elapsed time the plurality of images from a plurality of images imaging the moving subject successively; and
in the controlling of the display, the combined image and the motion data are displayed on the display with the time series of the plurality of images used in generating the combined image corresponded with the time series of the obtained motion data.

4. The exercise evaluating apparatus according to claim 3, wherein, in the controlling of the display, the combined image and the motion data are displayed on the display so that the time series of the plurality of images used in generating the combined image matches with the time series of the obtained motion data.

5. The exercise evaluating apparatus according to claim 1, wherein,
in the generating, the combined image is generated by overlapping the plurality of images from the plurality of images successively imaging the moving subject; and
in the controlling of the display, the combined image and the motion data are displayed on the display with the time series of the plurality of images used in generating the combined image corresponded with the time series of the obtained motion data.

6. The exercise evaluating apparatus according to claim 3, wherein,
the processor further performs first specifying to specify a predetermined point in the time series of the motion data displayed on the display; and
in the generating, when the point is specified in the first specifying, the combined image is generated positioning a specified image corresponding the point in the plurality of images as a center.

7. The exercise evaluating apparatus according to claim 3, wherein,
the processor further performs second specifying to specify a predetermined term in the time series of the motion data displayed on the display; and
in the generating, when the term is specified in the second specifying, the combined image is generated based on the plurality of images included in the term among the plurality of images.

8. The exercise evaluating apparatus according to claim 1, wherein,
the processor further performs first specifying to specify a predetermined point in the time series of the motion data displayed on the display; and
in the generating, the combined image is generated so that the subject of the image corresponding to the point specified in the first specifying is distinguished from the subject of the image other than the specified point in the plurality of images used to generate the combined image.

9. The exercise evaluating apparatus according to claim 6, wherein,
in the generating, the combined image is generated so that the subject of the image corresponding to the point specified in the first specifying is distinguished from the subject of the image other than the specified point in the plurality of images used to generate the combined image.

10. The exercise evaluating apparatus according to claim 1, wherein, in controlling the display, the plurality of combined images corresponding to each of the plurality of subjects and the plurality of motion data are displayed on the display.

11. The exercise evaluating apparatus according to claim 1,
wherein,
the processor further performs exercise analyzing based on the motion data; and
in the controlling of the display, the combined image and an exercise analysis result are displayed on the display with the time series of the plurality of images used in generating the combined image corresponded with a time series of the exercise analysis result.

12. The exercise evaluating apparatus according to claim 11, wherein,
the motion data includes acceleration data of a plurality of axis directions; and
the exercise analysis result includes a diagram showing a combined vector for each sample cycle of the motion data, the combined vector being a sum of acceleration vectors of a plurality of axes based on the acceleration data of the plurality of axis directions.

13. The exercise evaluating apparatus according to claim 11, wherein,
the motion data includes acceleration data of a plurality of axis directions; and
the exercise analysis result includes a diagram showing a displacement for each sample cycle of the motion data, the displacement in at least any of the plurality of axes of the subject.

14. The exercise evaluating apparatus according to claim 11, wherein,
the motion data includes angular speed data with the plurality of axis directions as the center; and
the exercise analysis result includes a diagram showing a rotating angle for each sample cycle of the motion data, the rotating angle in at least any of the plurality of axes of the subject.

15. An exercise evaluating method used in an exercise evaluating apparatus, the method comprising:
generating a combined image from a plurality of images successively imaging a moving subject, the combined image being an image in which a change in motion of the subject according to a time series can be understood;
obtaining motion data corresponding to motion of the subject output from a motion sensor attached to the subject; and
displaying on a display the combined image and the motion data in a state corresponding a time series of the plurality of images used in generating the combined image with a time series of the motion data.

16. A non-transitory computer-readable storage medium having a program stored thereon for controlling a computer of an exercise evaluating apparatus, wherein the program controls the program to perform:
generating a combined image from a plurality of images successively imaging a moving subject, the combined image being an image in which a change in motion of the subject according to a time series can be understood;
obtaining motion data corresponding to motion of the subject output from a motion sensor attached to the subject; and
displaying on a display the combined image and the motion data in a state corresponding a time series of the plurality of images used in generating the combined image with a time series of the motion data.
